(19) United States
(12) Patent Application Publication
(10) Pub. No.: US 2025/0303085 A1
(43) Pub. Date: Oct. 2, 2025

(54) MEDICAL DEVICE

(71) Applicant: FUKUDA DENSHI CO., LTD., Tokyo (JP)

(72) Inventors: Takanori ONO, Tokyo (JP); Naoto SHIRAISHI, Tokyo (JP)

(21) Appl. No.: 19/092,319

(22) Filed: Mar. 27, 2025

(30) Foreign Application Priority Data
Mar. 29, 2024 (JP) 2024-056573

(51) Int. Cl. A61M 16/00; A61M 16/10

(52) U.S. Cl. CPC A61M 16/024; A61M 16/0066; A61M 16/109; A61M 2205/215; A61M 2205/3327; A61M 2205/3379

(57) ABSTRACT

The medical device of the present disclosure includes an air blower that generates an airflow to be delivered to an airway of a patient, a sensor that detects acceleration or posture of the medical device, a water tank that retains water to be added to the airflow as moisture, and a controller that controls the operation of the medical device based on the acceleration or posture detected by the sensor.

### CROSS REFERENCE TO RELATED APPLICATIONS

This application is entitled to and claims the benefit of Japanese Patent Application No. 2024-056573, filed on Mar. 29, 2024, the disclosure of which including the specification, drawings and abstract is incorporated herein by reference in its entirety.

### TECHNICAL FIELD

The technology of the present disclosure relates to medical devices, such as devices for treating sleep apnea syndrome.

### BACKGROUND ART

Sleep apnea syndrome (SAS) is a condition with a state where breathing stops (apnea) and a state where breathing becomes weak (hypopnea) repeating intermittently during sleep. Patients suffering from sleep apnea syndrome often do not get enough sleep, leading to daytime drowsiness, decreased concentration, an increased risk of serious accidents due to drowsiness while driving, and the like. Most patients suffering from sleep apnea syndrome exhibit symptoms of obstructive sleep apnea (OSA). Obstructive sleep apnea occurs when muscle tone decreases during inhalation in sleep, causing the upper airway to narrow.

For patients with such obstructive sleep apnea, continuous positive airway pressure (CPAP) therapy may be administered. CPAP therapy is a treatment method that prevents apnea during sleep by continuously supplying air to an airway of a patient to keep the airway open.

Traditionally, devices that perform CPAP therapy are referred to as sleep apnea syndrome treatment devices or CPAP devices. Herein, devices that perform CPAP therapy will be referred to as CPAP devices. CPAP devices are described in, for example, PTL 1 below.

A CPAP device includes an air blower, a flow sensor, and a controller, and is configured to generate an airflow suitable for expanding an airway of a patient. Some CPAP devices include a water tank to humidify the airflow to be sent to a patient, preventing the airway of the patient from drying out.

Such a CPAP apparatus is described in, for example, Japanese Patent Application Laid-Open No. 2023-071739.

When a CPAP device is used for sleep, the lights in the room are usually turned off, making the room dark. Therefore, since the room is dark before sleep and upon waking, there is a possibility that the patient may accidentally bump into the CPAP device and cause it to fall over. Furthermore, the CPAP device may fall over due to, for example, the patient turning over in their sleep.

However, it cannot be said that sufficient consideration has been given to measures against these issues, and therefore, such measures have been insufficient in terms of safety and reliability.

The present disclosure has been made in consideration of the above circumstances and provides a medical device with improved safety and reliability.

### SUMMARY

One aspect of the medical device of the present disclosure includes:
an air blower that generates an airflow to be delivered to an airway of a patient; and
a sensor that detects acceleration or posture of the medical device.

### BRIEF DESCRIPTION OF THE DRAWINGS

FIG. 1 illustrates a CPAP device being attached to a patient;

FIG. 2A is a perspective view of the CPAP device from diagonally above;

FIG. 2B is a perspective view of the CPAP device from diagonally above;

FIG. 3 is an exploded perspective view of the CPAP device according to an embodiment;

FIG. 4 is a schematic diagram illustrating the path of an airflow;

FIG. 5 is a block diagram for explaining the configuration of the CPAP device according to the embodiment;

FIG. 6 is a flowchart illustrating a flow of control using an acceleration sensor according to the embodiment;

FIGS. 7A and 7B are diagrams for explaining a case where the CPAP device is dropped without being tilted, FIG. 7A illustrates a state where the CPAP device is dropped without being tilted, and FIG. 7B illustrates a Z-axis component output from the acceleration sensor at that time;

FIGS. 8A and 8B are diagrams for explaining a case where the CPAP device is tilted and dropped, FIG. 8A illustrates a state where the CPAP device is tilted and dropped, and FIG. 8B illustrates the X-axis and/or Y-axis component output from the acceleration sensor at that time;

FIG. 9A illustrates the X-axis and/or Y-axis component output from the acceleration sensor when the CPAP device falls over;

FIG. 9B illustrates the X-axis and/or Y-axis component output from the acceleration sensor when the CPAP device receives an impact;

FIG. 9C illustrates the X-axis and/or Y-axis component output from the acceleration sensor when the CPAP device receives vibration;

FIGS. 10A to 10C are diagrams for explaining examples where a two-axis acceleration sensor can be used; and

FIGS. 11A and 11B are diagrams for explaining examples where a two-axis acceleration sensor can be used.

### DESCRIPTION OF EMBODIMENTS

Hereinafter, embodiments of the present disclosure will be described in detail with reference to the drawings.

### <1> Configuration of CPAP Device According to Embodiment

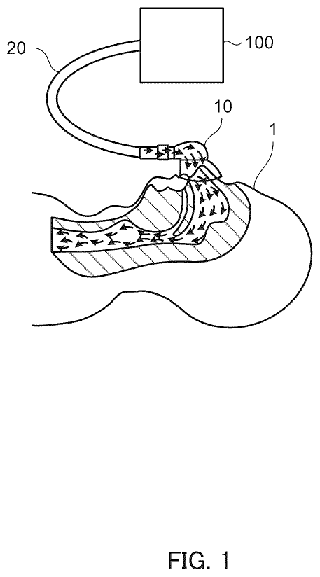

As illustrated in FIG. 1, CPAP device 100 is connected via tube 20 to mask 10 worn on the face of patient 1 suffering from sleep apnea syndrome, and delivers an airflow with a positive pressure to the upper airway of patient 1 to expand the upper airway. In the present embodiment, CPAP device 100 represents the main body of the CPAP device, and the CPAP device is configured to include CPAP device 100 as the CPAP device main body, mask 10, and tube 20.

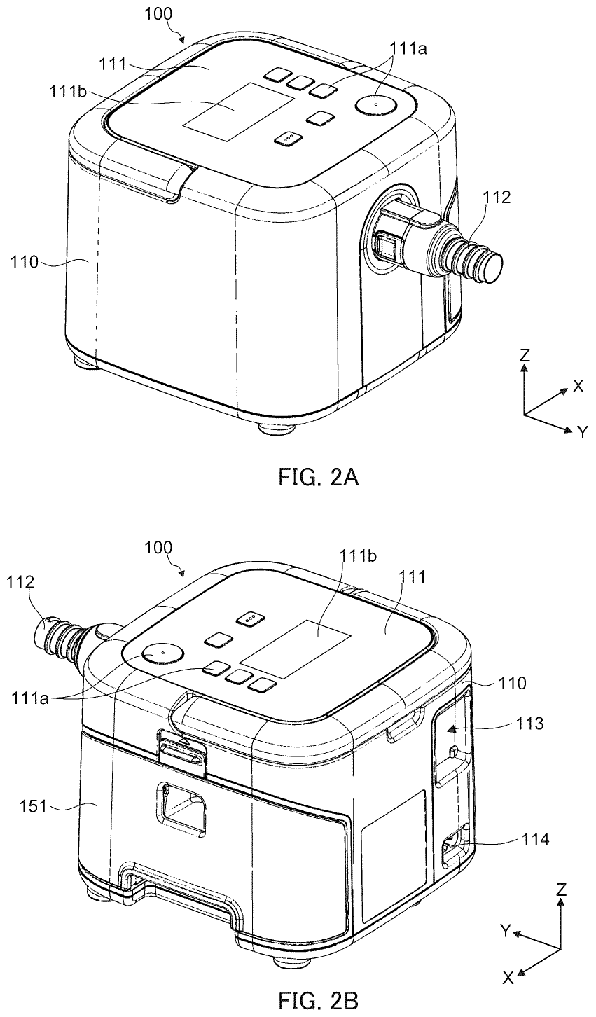

FIGS. 2A and 2B are perspective views of CPAP device 100 from diagonally above. Here, the +Z direction in the drawing indicates the upward direction of CPAP device 100, and the −Z direction indicates the downward direction of CPAP device 100. In addition, the +Y direction indicates the forward direction of CPAP device 100, and the −Y direction indicates the backward direction of CPAP device 100. Furthermore, the +X direction indicates the left direction of CPAP device 100, and the −X direction indicates the left direction of CPAP device 100.

As can be seen from FIG. 2A, tube connector 112, to which tube 20 (FIG. 1) is connected, protrudes from the front side surface of housing case 110 of CPAP device 100. In addition, operation panel 111 is provided on the upper part of housing case 110. Operation panel 111 is provided with operation input 111a including operation buttons, and display 111b.

As can be seen from FIG. 2B, intake port 113 and power connector 114 are provided on the rear side surface of housing case 110. Power connector 114 receives an AC power supply via a power cable. In addition, water tank 151 is detachably attached to the side surface of housing case 110.

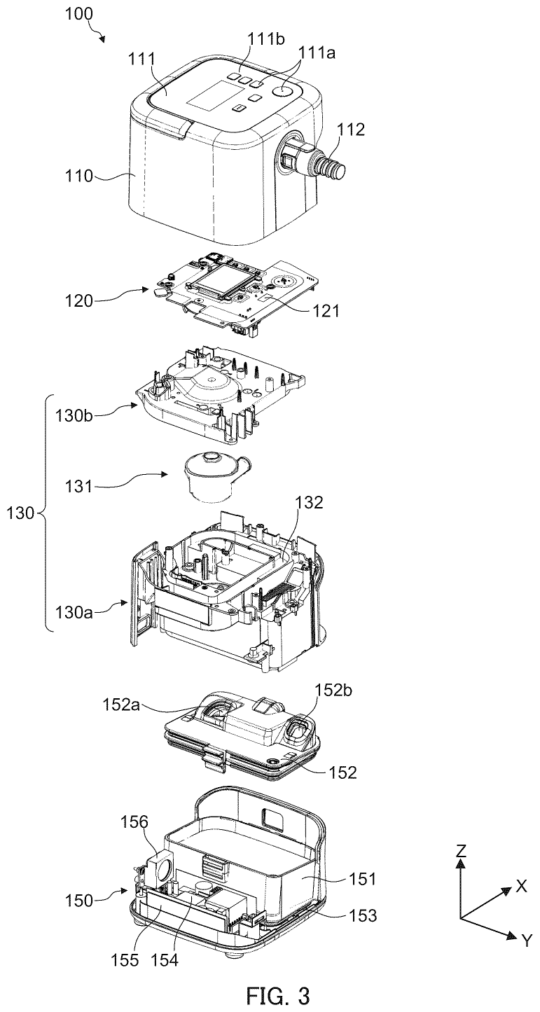

FIG. 3 is an exploded perspective view of CPAP device 100 according to the present embodiment.

CPAP device 100 mainly includes housing case 110, circuit board 120, flow path case 130, and base 150.

Housing case 110 has a rectangular cylindrical shape and houses circuit board 120, flow path case 130, and the like by being coupled from above to base 150.

Circuit board 120 is provided with a central processing unit (CPU), various driver circuits, and the like. In addition, in the present embodiment, circuit board 120 is provided with acceleration sensor 121.

Flow path case 130 is configured by fitting a lower case 130a and an upper case 130b together. Inside flow path case 130, blower 131 as the first blower is disposed. Flow path 132, through which the wind generated by blower 131 passes, is formed inside flow path case 130.

A detachable water tank 151 is disposed on base 150. Air inlet 152a and air outlet 152b are formed on lid 152 of water tank 151. Air inlet 152a communicates with flow path 132 located inside flow path case 130. Air outlet 152b communicates with tube connector 112.

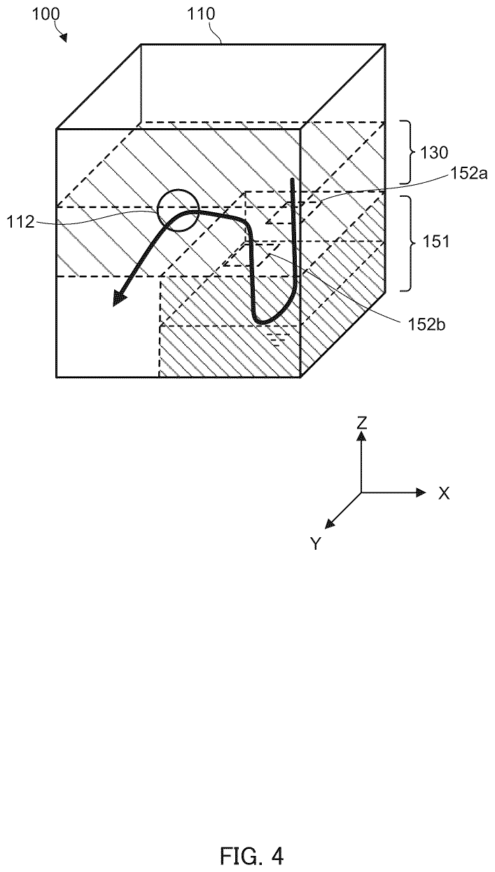

As can be seen from the schematic diagram in FIG. 4, an airflow (indicated by arrows in the drawing) generated by blower 131 thus passes through flow path 132 of flow path case 130 (FIG. 3), enters water tank 151 through air inlet 152a, is discharged from water tank 151 through air outlet 152b, and is supplied to the patient via tube connector 112.

Heater 153 is provided on the lower surface side of water tank 151. The water in water tank 151 is heated by heater 153 to create a high humidity state inside water tank 151. Therefore, the airflow to be supplied to the patient is humidified inside water tank 151. This reduces the drying of the airway of patient 1 by the airflow.

In addition, base 150 is provided with AC/DC converter 154. AC/DC converter 154 receives AC power from the outside through a power cord (not illustrated) connected to power connector 114 (FIG. 2B), converts the power to DC power, and supplies the DC power obtained by the conversion to circuit board 120 and the like.

A plurality of circuit components constituting AC/DC converter 154 are covered from the bottom and both left and right sides by sheet metal member 155 that has a U-shaped cross section taken along the XZ plane. Sheet metal member 155 extends in the Y direction. At one end of sheet metal member 155, fan 156 is provided as a second air blower to cool AC/DC converter 154. Fan 156 is provided at a position to face AC/DC converter 154.

Thus, AC/DC converter 154 is efficiently cooled by the wind from fan 156, flowing inside sheet metal member 155 in the extending direction of sheet metal member 155. In addition, the electromagnetic noise generated by AC/DC converter 154 is blocked by sheet metal member 155.

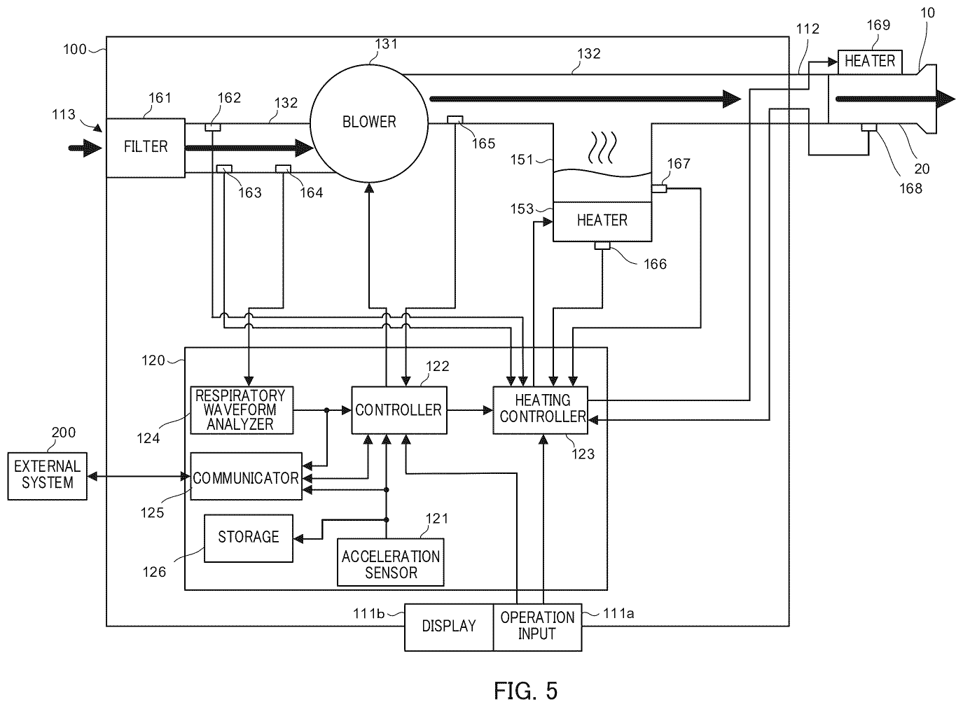

FIG. 5 is a block diagram for explaining the configuration of CPAP device 100.

In addition to blower 131, filter 161, temperature sensor 162, humidity sensor 163, flow sensor 164, and pressure sensor 165 are provided at flow path 132 of CPAP device 100. In addition, temperature sensor 166 is attached to heater 153 that heats water tank 151, and a weight sensor 167 is attached to water tank 151.

Circuit board 120 is provided with acceleration sensor 121, controller 122, heating controller 123, respiratory waveform analyzer 124, communicator 125, storage 126, and the like. In other words, circuit board 120 is equipped with circuit components to realize the functions of acceleration sensor 121, controller 122, heating controller 123, respiratory waveform analyzer 124, communicator 125, and storage 126.

Controller 122, heating controller 123, and respiratory waveform analyzer 124 include a central processing unit (CPU), read only memory (ROM), random access memory (RAM), and the like. The CPU reads out a program corresponding to the processing content from the ROM and deploys the program in the RAM, and cooperates with the loaded program to realize the functions of controller 122, heating controller 123, and respiratory waveform analyzer 124. All or part of controller 122, heating controller 123, and respiratory waveform analyzer 124 may be formed from hardwired circuits such as an application specific integrated circuit (ASIC) or a field-programmable gate array (FPGA).

When blower 131 operates, external air enters flow path 132 through intake port 113 and filter 161. The temperature and humidity of the air in flow path 132 are measured by temperature sensor 162 and humidity sensor 163, and the measured temperature and humidity are sent to heating controller 123. Furthermore, heating controller 123 receives the heating set value and humidification set value (e.g., target temperature and target humidity) from operation input 111a, along with the temperature information of heater 153 from temperature sensor 166.

Heating controller 123 controls heater 153 based on the temperature and humidity information measured by temperature sensor 162 and humidity sensor 163, the heating set value and humidification set value from operation input 111a set by the user, and the temperature information of heater 153 from temperature sensor 166. For example, heating controller 123 controls heater 153 so that the temperature and humidity of the airflow to be supplied to patient 1 approach the heating set value and humidification set value.

In addition, heating controller 123 receives temperature information from temperature sensor 168 provided at tube 20. Heating controller 123 controls heater 169 provided at tube 20 based on this temperature information to prevent condensation within tube 20.

Flow sensor 164 is a differential pressure sensor that measures the respiratory flow of patient 1 and sends the measurement result to respiratory waveform analyzer 124. Respiratory waveform analyzer 124 performs analyze to acquire the respiratory waveform of patient 1 based on the respiratory flow and sends the acquired respiratory waveform as respiratory information to controller 122 and communicator 125.

The information on the pressure in flow path 132 measured by pressure sensor 165 is sent to controller 122. In addition, controller 122 receives pressure setting information (e.g., target pressure) from operation input 111a. Controller 122 controls the rotation of blower 131 based on the pressure information measured by pressure sensor 165, the respiratory information from respiratory waveform analyzer 124, and the pressure setting information from operation input 111a set by the user, thereby controlling the pressure of the airflow supplied to patient 1.

The acceleration information (detected value) measured by acceleration sensor 121 is sent to controller 122, communicator 125, and storage 126.

Controller 122 controls the operation of CPAP device 100 based on the acceleration information. Controller 122 performs determinations regarding CPAP device 100, such as drop, fall, impact, and vibration, based on the acceleration information, and when controller 122 determines that CPAP device 100 has dropped, fallen, been impacted, or vibrated, controller 122 stops the operation of all or part of CPAP device 100.

Communicator 125 communicates with external system 200. For example, the respiratory information obtained by respiratory waveform analyzer 124 is transmitted to external system 200 via communicator 125. This allows medical personnel at a distance from CPAP device 100 to know that patient 1 is experiencing apnea.

In addition, the acceleration obtained by acceleration sensor 121, the determination results regarding the drop, fall, impact, and vibration obtained by controller 122, and the operation stop information performed by controller 122 are transmitted to external system 200 via communicator 125. This allows the acceleration that occurred in CPAP device 100, drop, fall, impact, vibration, and operation stop to be known at external system 200. As a result, when external system 200 is a system server of a management company, such information can be utilized for maintenance work of CPAP device 100.

### <2> Processing Using Acceleration Sensor

Next, a processing example using acceleration sensor 121 in the present embodiment will be specifically described.

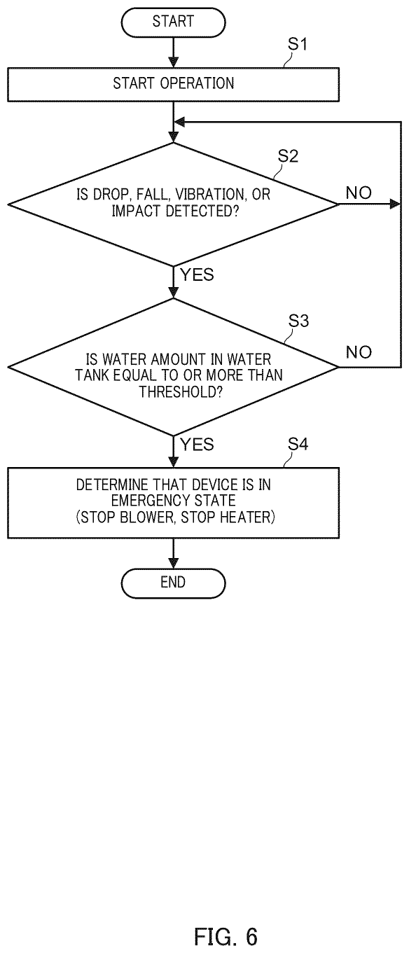

FIG. 6 is a flowchart illustrating the control flow using acceleration sensor 121 according to the present embodiment. In step S1, controller 122 starts the operation of CPAP device 100 by controlling the drive of blower 131, heater 153, and the like. After starting the operation, controller 122 continuously or periodically measures the amount of water (i.e., water amount) in water tank 151 and stores the information.

In the subsequent step S2, controller 122 determines whether a drop, fall, vibration, or impact of CPAP device 100 is detected. Specifically, controller 122 determines whether CPAP device 100 has dropped, fallen, vibrated at a predetermined value or more, or received an impact at a predetermined value or more, based on the acceleration information from acceleration sensor 121.

When a positive result is obtained in step S2 (step S2; YES), controller 122 proceeds to step S3. In step S3, controller 122 determines whether the amount of water in water tank 151 is equal to or more than a threshold. In the example of the present embodiment, controller 122 estimates the amount of water in water tank 151 based on the measurement results of the weight sensor 167 and performs threshold determination.

When a positive result is obtained in step S3 (step S3; YES), controller 122 proceeds to step S4. In step S4, controller 122 determines that CPAP device 100 is in an emergency state and stops blower 131 and heaters 153 and 169.

By performing the control as illustrated in FIG. 6, even when CPAP device 100 drops, falls over, or sways violently, and water overflowing from water tank 151 enters tube 20, the water is not pumped to patient 1 by the blower, thus the water overflowing from the water tank 151 is prevented from being mistakenly sent to the airway of patient 1. In addition, when CPAP device 100 drops, falls over, or sways violently, heaters 153 and 169 are stopped, which prevents damage to heaters 153 and 169 and burns to the user that would be caused by overheating of the heaters 153 and 169 due to empty heating or the like.

It is also possible to proceed to step S4 without performing the processing of step S3 when a positive result is obtained in step S2.

This configuration allows for the realization of CPAP device 100 with improved safety and reliability.

In countries like Japan, where it is customary to sleep on the floor, CPAP device 100 is often placed on the floor, posing a risk that a patient or family members may accidentally kick CPAP device 100, causing it to fall over. There is also a risk that CPAP device 100 may fall over due to, for example, tossing and turning in sleep. On the other hand, in countries like those in Europe and America, where it is customary to sleep in beds, CPAP device 100 is often placed higher than the floor, posing a risk of CPAP device 100 being dropped. Furthermore, in countries with many earthquakes, there is a risk that CPAP device 100 may drop, fall over, or sway violently due to an earthquake.

In such cases, CPAP device 100 of the present embodiment can prevent water from entering tube 20, thereby preventing clogging of tube 20 by water. Even when water enters tube 20, the water can be prevented from being sent toward mask 10. Furthermore, overheating of heaters 153 and 169 can be prevented, thus avoiding damage to heaters 153 and 169 and burns to the user.

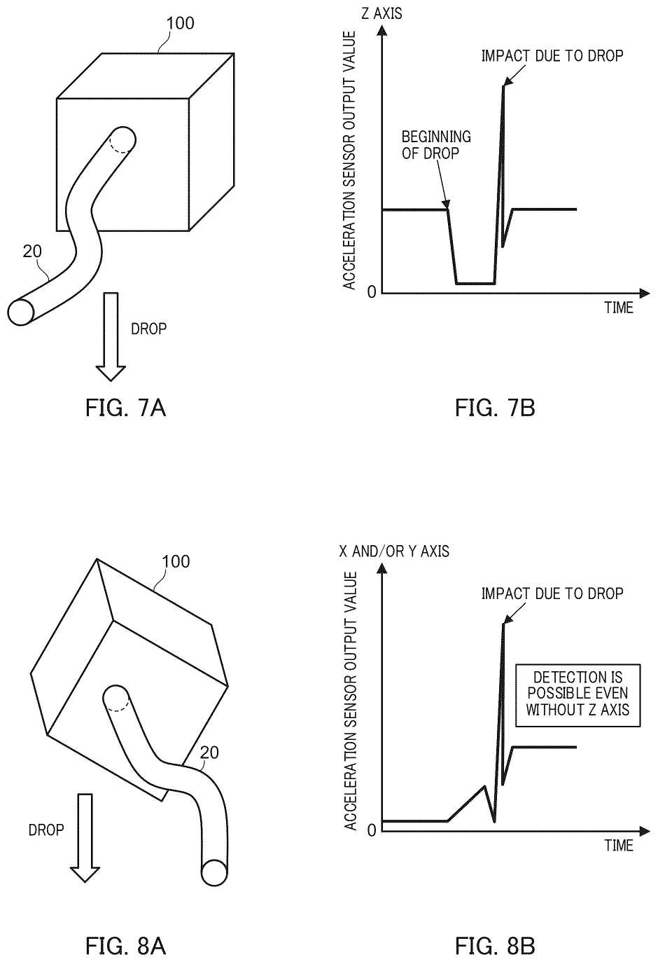

Here, using FIGS. 7 to 9, the detection of the drop, impact, fall, and vibration using acceleration information will be described.

FIGS. 7A to and 8B are diagrams for explaining a drop and the impact due to the drop. FIGS. 7A and 7B are diagrams for explaining a case where CPAP device 100 is dropped without being tilted, and FIGS. 8A and 8B are diagrams for explaining a case where CPAP device 100 is tilted and dropped.

FIG. 7A illustrates the state where CPAP device 100 is dropped without being tilted, and FIG. 7B illustrates the Z-axis component output from acceleration sensor 121 at that time. As can be seen from FIG. 7B, the drop of CPAP device 100 and the impact on CPAP device 100 due to the drop can be detected from the positions where the Z-axis component changes significantly. Note that in FIG. 7B, gravitational acceleration is always applied in the Z-axis direction.

FIG. 8A illustrates the state where CPAP device 100 is tilted and dropped, and FIG. 8B illustrates the X-axis and/or Y-axis component output from acceleration sensor 121 at that time. As can be seen from FIG. 8B, the impact on CPAP device 100 due to the drop can be detected from the position where the X-axis and/or Y-axis component changes significantly. In other words, the impact due to a drop can be detected even without the Z-axis component. Therefore, even when a two-axis or one-axis acceleration sensor is used instead of a three-axis acceleration sensor as acceleration sensor 121, the impact due to a drop can be detected. By using a two-axis or one-axis acceleration sensor, there is an advantage that a simpler and less expensive configuration can be used for acceleration sensor 121.

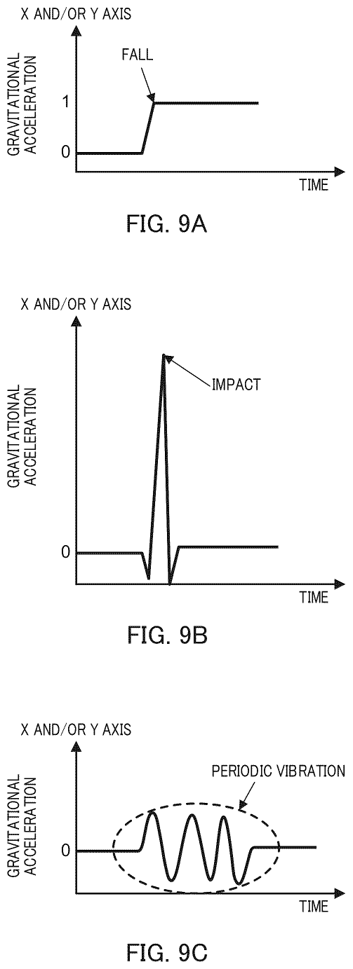

FIG. 9A illustrates the X-axis and/or Y-axis component output from acceleration sensor 121 when CPAP device 100 falls over. FIG. 9B illustrates the X-axis and/or Y-axis component output from acceleration sensor 121 when CPAP device 100 receives an impact (for example, when CPAP device 100 placed on the floor is kicked by a foot). FIG. 9C illustrates the X-axis and/or Y-axis component output from acceleration sensor 121 when CPAP device 100 receives vibrations, for example, due to an earthquake.

Controller 122 determines the drop, impact, fall, and vibration of CPAP device 100 by analyzing which of the waveform shapes in FIG. 7B, FIG. 8B, FIG. 9A, FIG. 9B, and FIG. 9C the output of acceleration sensor 121 corresponds to.

Then, controller 122 stops the operation of all or part of CPAP device 100 when controller 122 determines, for example, that CPAP device 100 has fallen over. In addition, when controller 122 determines that CPAP device 100 has received an impact or is vibrating, controller 122 stops the operation of all or part of CPAP device 100 when the magnitude of the impact or vibration is equal to or more than a threshold.

Furthermore, in one embodiment, the following configuration is possible: controller 122 stops the operation of all or part of CPAP device 100 when controller 122 determines that CPAP device 100 has fallen over, but does not perform stop control when controller 122 determines that CPAP device 100 has received an impact or is vibrating. In other words, stop control of CPAP device 100 is performed when CPAP device 100 falls over because the safety of CPAP device 100 significantly decreases in this case, whereas the stop control is not performed when CPAP device 100 simply receives an impact or is vibrating because the decrease in safety is smaller in this case than when CPAP device 100 falls over. This approach prevents CPAP device 100 from being stopped unnecessarily.

when

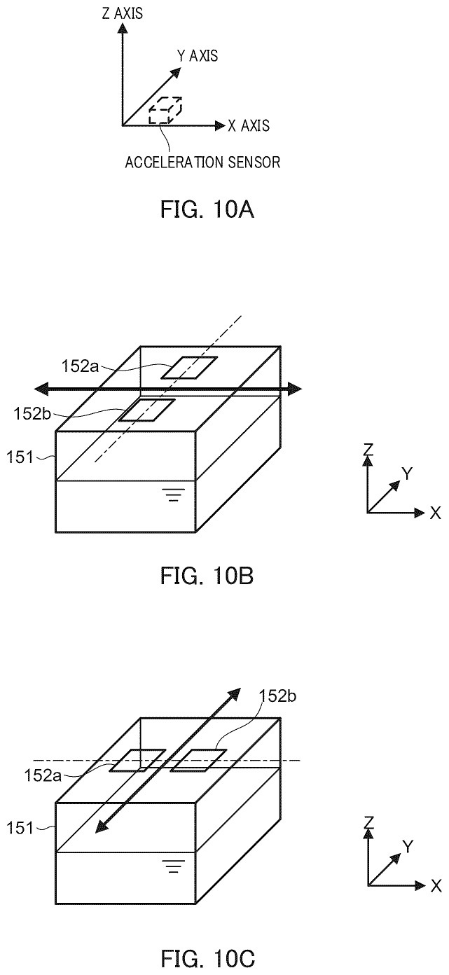

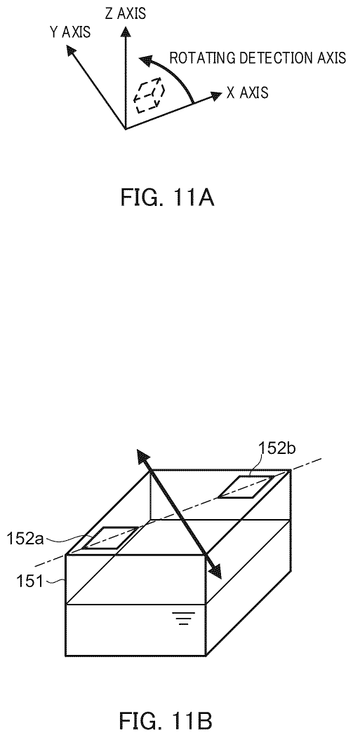

Next, an example in which a two-axis acceleration sensor, which has a simpler configuration than a three-axis acceleration sensor, can be used as acceleration sensor 121 will be described with reference to FIGS. 10 and 11.

FIG. 10A illustrates the detection axes of the acceleration sensor. Here, the detection axes of a three-axis acceleration sensor are the X, Y, and Z axes, those of a two-axis acceleration sensor are the X and Y axes, and that of a one-axis acceleration sensor is the X axis. For example, as illustrated in FIGS. 10B and 10C, when air inlet 152a and air outlet 152b on the top surface of water tank 151 are aligned along the direction indicated by the dash-dotted line in the drawing, it is preferable to detect acceleration in the direction indicated by the dash-dotted line. In other words, in FIGS. 10B and 10C, it is preferable to dispose an acceleration sensor in such a way that the detection axis (X-axis) of the acceleration sensor is along the dash-dotted line, regardless of the number of detection axes. By doing so, regardless of the number of detection axes, even a one-axis acceleration sensor can detect water leakage from air inlet 152a and air outlet 152b.

When the center line (indicated by the dash-dotted line in the drawing) of air inlet 152a and air outlet 152b on the top surface of water tank 151 is as illustrated in FIG. 11B, by rotating the detection axis of an acceleration sensor to align the detection axis with the center line as illustrated in FIG. 11A, it is possible to eliminate the detection of acceleration in the direction perpendicular to the direction of the center line (the direction of the arrow in the drawing).

### <3> Summary

As described above, according to one aspect of the embodiment of CPAP device 100, CPAP device 100 includes an air blower (blower 131) that generates an airflow to be delivered to an airway of a patient, acceleration sensor 121, water tank 151 that retains water to be added to the airflow as moisture, and controller 122 that controls the operation of CPAP device 100 based on acceleration detected by acceleration sensor 121. This enables the realization of CPAP device 100 with improved safety and reliability.

In addition, according to one aspect of the embodiment of CPAP device 100, controller 122 determines whether CPAP device 100 has fallen over based on the acceleration, and when a determination result indicating that CPAP device 100 has fallen over is obtained, controller 122 stops the operation of all or part of CPAP device 100. This prevents water overflowing from water tank 151 from being accidentally sent toward mask 10 when CPAP device 100 has fallen over by, for example, stopping the operation of blower 131.

Furthermore, according to one aspect of the embodiment of CPAP device 100, controller 122 controls the operation of CPAP device 100 based on the acceleration and the amount of water in water tank 151. In addition, according to one aspect of the embodiment of CPAP device 100, controller 122 stops the operation of all or part of CPAP device 100 when the detected value of the acceleration is equal to or more than a predetermined value and the detected value of the amount of water is equal to or more than a predetermined value. This makes it possible to prevent the operation of CPAP device 100 from being stopped when the acceleration is equal to or more than a threshold but the amount of water is less than a threshold, thereby minimizing unnecessary operation stop control.

Moreover, according to one aspect of the embodiment of CPAP device 100, acceleration sensor 121 is provided at a position above water tank 151. In the above embodiment, acceleration sensor 121 is provided on circuit board 120 above water tank 151. In this way, when CPAP device 100 receives an impact or sways, the impact and swaying received by water tank 151 can be detected with high sensitivity by acceleration sensor 121, enabling more reliable and safe operation stop control.

Furthermore, the installation position of acceleration sensor 121 is not limited to the above position. Acceleration sensor 121 may be positioned, for example, at the center of gravity or center position of CPAP device 100, or at the center of gravity or center position of water tank 151. Alternatively, acceleration sensor 121 may be positioned, for example, higher or lower than the center of gravity or center position of CPAP device 100, or higher or lower than the center of gravity or center position of water tank 151.

Moreover, according to one aspect of the embodiment of CPAP device 100, CPAP device 100 includes storage 126 that stores information related to acceleration. Here, information related to acceleration includes the acceleration itself, or determination results regarding the drop, fall, impact, and vibration, as well as information on the stop of operation. This allows an administrator to recognize whether a malfunction, such as a failure in CPAP device 100, is caused by the drop, fall, impact, and vibration of CPAP device 100 by checking the information related to acceleration stored in storage 126. Furthermore, information related to acceleration may be stored in association with time. In this way, the administrator can recognize when the drop, fall, impact, and vibration occurred.

In addition, according to one aspect of the embodiment of CPAP device 100, CPAP device 100 includes communicator 125 that transmits information related to acceleration to the outside of the device. This allows the outside parties to know the acceleration that occurred in CPAP device 100, drop, fall, impact, vibration, and operation stop. As a result, for example, the maintainability of CPAP device 100 is improved.

The embodiments described above are merely examples of specific implementations of the present invention, and should not be construed as limiting the technical scope of the present invention. That is, the present invention can be implemented in various forms without departing from the spirit or main features thereof.

In the example of the embodiment described above, a case has been described where the amount of water in water tank 151 is estimated based on the measurement result of weight sensor 167, but the present disclosure is not limited thereto. The amount of water may be estimated, without providing the weight sensor 167, based on the heating time of heater 153, based on the heating time and temperature of heater 153, or based on the power consumption of heater 153, or the amount of water may be measured using a water level sensor. Alternatively other methods may be used for the estimation.

In the above embodiment, a case where acceleration sensor 121 is provided has been described, but instead of or in addition to acceleration sensor 121, a posture sensor may be provided. For example, the falling over of CPAP device 100 may be detected by the posture sensor. For example, when the posture sensor detects that CPAP device 100 has tilted from a horizontal position, an alert may be output an instruction to place the device on a horizontal surface.

As a posture sensor, a sensor capable of detecting the installation state of CPAP device 100 such as the tilt of the device, for example, a gyro sensor, angle sensor, tilt sensor, or angular acceleration sensor, can be used.

However, using an acceleration sensor as in the above embodiment is preferable because all of the drop, fall, impact, and vibration of CPAP device 100 can be detected.

Furthermore, in addition to the configuration of the above embodiment, the following configuration is possible: a sensor to detect water entering flow path 132 is provided, and when water enters flow path 132, an alert is output, or the operation of blower 131 and the like is stopped. In this way, safety and reliability can be further enhanced.

In the above embodiment, a case has been described where the present invention is applied to a CPAP device with water tank 151, but the present invention can also be applied to a CPAP device without a water tank. That is, even in a CPAP device without a water tank, when the drop, fall, impact, and/or vibration occurs, tube 20 may bend, or the device may malfunction, leading to decreased safety and reliability. When acceleration sensor 121 is provided in the CPAP device, the acceleration sensor can detect the drop, fall, impact, and/or vibration, and when these are detected, controller 122 can perform operation stop control, transmission to the outside via communicator 125, or storing in storage 126, thereby improving safety and reliability.

Furthermore, the technology of the present disclosure is not limited to CPAP devices, but can also be applied to respiratory support devices such as adaptive servo ventilation (ASV) devices and nasal high flow (NHF) devices as well as to other home medical devices.

(1) One aspect of the medical device of the present disclosure includes: an air blower that generates an airflow to be delivered to an airway of a patient; and a sensor that detects acceleration or posture of the medical device.

As a result, the detection of the drop, impact, fall, vibration, and the like becomes possible, and therefore, medical devices with improved safety and reliability are realized.

(2) One aspect of the medical device of the present disclosure, in (1) above, further includes: a water tank that retains water to be added to the airflow as moisture; and a controller that controls the operation of the medical device based on the result of detection by the sensor.

As a result, adverse effects on the medical device caused by the water in the water tank can be prevented.

(3) In one aspect of the medical device of the present disclosure in (2) above, the controller determines whether the medical device has fallen over based on the result of the detection, and when a determination result indicating that the medical device has fallen over is obtained, the controller stops the operation of all or part of the medical device.

As a result, adverse effects on the medical device caused by water in the water tank when the medical device falls over can be prevented.

(4) In one aspect of the medical device of the present disclosure in (2) above, the controller controls the operation of the medical device based on the acceleration and the water amount in the water tank.

(5) In one aspect of the medical device of the present disclosure in (4) above, the controller stops the operation of all or part of the medical device when the detected value of the acceleration is equal to or more than a predetermined value and the detected value of the water amount is equal to or more than a predetermined value.

As a result, for example, even when the acceleration is equal to or more than a threshold, but the water amount is less than a threshold, the operation of the medical device is not stopped, and therefore unnecessary operation stop control of the medical device can be prevented.

(6) One aspect of the medical device of the present disclosure, in (3) or (5) above, a component of the all or part of the medical device whose operation is stopped by the controller includes the air blower.

(7) One aspect of the medical device of the present disclosure, in (3) or (5) above, further includes a heater that heats the water in the water tank, and a component of the all or part of the medical device whose operation is stopped by the controller includes the heater.

(8) In one aspect of the medical device of the present disclosure in (2) above, the acceleration sensor is provided above the water tank.

(9) One aspect of the medical device of the present disclosure, in (1) above, further includes a storage that stores information related to the acceleration.

(10) One aspect of the medical device of the present disclosure, in (1) above, further includes a communicator that transmits information related to the acceleration to an outside of the medical device.

### REFERENCE SIGNS LIST

1 Patient
10 Mask
20 Tube
100 CPAP device
110 Housing case
111 Operation panel
112 Tube connector
113 Intake port
114 Power connector
120 Circuit board
121 Acceleration sensor
130 Flow path case
131 Blower
132 Flow path
150 Base
151 Water tank
152 Lid
152a Air inlet
152b Air outlet
153, 169 Heater
154 AC/DC converter
155 Sheet metal member
156 Fan
122 Controller
123 Heating controller
125 Communicator
126 Storage

1. A medical device comprising:
an air blower that generates an airflow to be delivered to an airway of a patient; and
a sensor that detects acceleration or posture of the medical device.

2. The medical device according to claim 1, further comprising:
a water tank that retains water to be added to the airflow as moisture; and
a controller that controls an operation of the medical device based on a result of detection by the sensor.

3. The medical device according to claim 2, wherein
the controller determines whether the medical device has fallen over based on the result of the detection, and when a determination result indicating that the medical device has fallen over is obtained, the controller stops the operation of all or part of the medical device.

4. The medical device according to claim 2, wherein
the controller controls the operation of the medical device based on the acceleration and a water amount in the water tank.

5. The medical device according to claim 4, wherein
the controller stops the operation of all or part of the medical device when a detected value of the acceleration is equal to or more than a predetermined value and a detected value of the water amount is equal to or more than a predetermined value.

6. The medical device according to claim 3, wherein
a component of the all or part of the medical device whose operation is stopped by the controller includes the air blower.

7. The medical device according to claim 3, further comprising:
a heater that heats the water in the water tank, wherein
a component of the all or part of the medical device whose operation is stopped by the controller includes the heater.

8. The medical device according to claim 2, wherein
the sensor includes an acceleration sensor, and the acceleration sensor is provided above the water tank.

9. The medical device according to claim 1, further comprising a storage that stores information related to the acceleration.

10. The medical device according to claim 1, further comprising a communicator that transmits information related to the acceleration to an outside of the medical device.
